(19) United States
(12) Patent Application Publication
(10) Pub. No.: US 2021/0217526 A1
(43) Pub. Date: Jul. 15, 2021

(54) CANCER DIAGNOSIS USING OPTIMAL CLUSTERING WITH SUCCESSIVE DECONVOLUTION

(71) Applicant: HIGHLAND INNOVATIONS INC., West New York, NJ (US)

(72) Inventors: Eung Joon Jo, West New York, NJ (US); Yohahn Jo, West New York, NJ (US)

(73) Assignee: HIGHLAND INNOVATIONS INC., West New York, NJ (US)

(21) Appl. No.: 17/145,385

(22) Filed: Jan. 10, 2021

Related U.S. Application Data
(60) Provisional application No. 62/959,219, filed on Jan. 10, 2020.
(60) Provisional application No. 62/959,223, filed on Jan. 10, 2020.

(51) Int. Cl. G16H 50/20; G06N 3/04; G16H 70/00; G16H 50/70

(52) U.S. Cl. CPC G16H 50/20; G16H 50/70; G16H 70/00; G06N 3/04

(57) ABSTRACT

An apparatus and/or method that includes deconvolving a pre-deconvoluted distribution of spectrometry reference profile peaks into at least two post-deconvoluted distributions of spectrometry reference profile peaks. Through this deconvolution, sufficiently narrow probability distribution functions may be attained, which may contribute to diagnostic accuracy. The pre-convoluted distribution of spectrometry reference profile peaks originated from spectrometry reference profiles associated with a first category. The at least two post-deconvoluted distributions of spectrometry reference profile peaks each originated from spectrometry reference profiles each associated with at least two different sub-categories of the first category. For example, the first category may be cancer in general and the at least two sub-categories may be types of cancer. These different types of cancer may be further deconvolved into more subcategories to form a cluster of probability distribution functions, which are meaningful in diagnostic applications.

The present application claims priority to U.S. Provisional Patent Application No. 62/959,219 filed on Jan. 10, 2020 and U.S. Provisional Patent Application No. 62/959,223 filed on Jan. 10, 2020, which are all hereby incorporated by reference in their entireties.

### BACKGROUND

Mass spectrometry may be used to diagnose diseases or in other non-medical applications. A sample of a target to be diagnosed (or otherwise identified) may be tested by a mass spectrometer that produces a mass spectrometry profile. The mass spectrometry profile may include one or more peaks at different mass-to-charge units or other measurement unit. These peaks are representative of the physical attributes of the sample of the target. Although these peaks do not contain any diagnostic information by themselves, they can be compared to a reference database of previously tested targets that do have known characteristic patterns. However, in order to generate any meaningful diagnostic information, the reference database must include probability distribution functions of attributes in sufficiently narrow ranges. Reference databases with overly wide distributions may prevent accurate diagnosis of diseases or other types of non-medical determinations.

### SUMMARY

Embodiments relate to an apparatus and/or method that includes deconvolving a pre-deconvoluted distribution of spectrometry reference profile peaks into at least two post-deconvoluted distributions of spectrometry reference profile peaks. Through this deconvolution, sufficiently narrow probability distribution functions may be attained, which may contribute to diagnostic accuracy. In embodiments, the pre-convoluted distribution of spectrometry reference profile peaks originated from spectrometry reference profiles associated with a first category. In embodiments, the at least two post-deconvoluted distributions of spectrometry reference profile peaks each originated from spectrometry reference profiles each associated with at least two different sub-categories of the first category. For example, the first category may be a particular cancer and the at least two sub-categories may be age groups. These different types of cancer may be further deconvolved into more subcategories to form a cluster of probability distribution functions, which are meaningful in diagnostic applications.

### DRAWINGS

Example FIG. 1 is a schematic view of a MALDI-TOF MS system, in accordance with embodiments.

Example FIG. 2 is a system diagram of the integrated system including a sample processing unit, a MALDI-TOF MS unit, and a diagnosis unit in one system, in accordance with embodiments.

Example FIG. 3 is a system diagram of an integrated diagnostic system including a sample processing unit and a MALDI-TOF MS unit integrated in one system, whereas a diagnosis unit is provided as a separate unit, in accordance with embodiments.

Example FIG. 4 is a MALDI-TOF MS hardware diagram, in accordance with embodiments.

FIG. 5 is an example MALDI-TOF mass spectra, in accordance with embodiments.

FIG. 6A illustrates example PCA plot of a mass spectrum before optimal clustering, in accordance with embodiments.

FIG. 6B illustrates an example PCA plot of a mass spectrum after optimal clustering in accordance embodiments.

Example FIG. 7 illustrates a system for matching characteristic information, in accordance with embodiments.

Example FIG. 8 illustrates a system for matching characteristic information using artificial intelligence, in accordance with embodiments.

FIG. 9-12 illustrate an example probability density function (PDF) including successive deconvolution, in accordance with embodiments.

### DESCRIPTION

As the invention allows for various changes and numerous embodiments, particular embodiments will be illustrated in the drawings and described in detail in the written description. However, this is not intended to limit the present invention to specific embodiments, it should be understood to include all modifications, equivalents, and substitutes included in the spirit and scope of the present invention. In describing the drawings, similar reference numerals may used for similar elements in a non-limiting fashion.

The terminology used herein is for the purpose of describing particular example embodiments only and is not intended to be limiting of the present invention or the claims. Singular expressions include plural expressions unless the context clearly indicates otherwise. In this application, the terms comprise or have are intended to indicate that there is a feature, number, step, operation, component, part, or combination thereof described in the specification, and one or more other features. It is to be understood that the present invention does not exclude the possibility of the presence or the addition of numbers, steps, operations, components, components, or a combination thereof.

Medical diagnosis is becoming increasingly important. Early detection of diseases greatly increases the changes for successful treatment. Recently, mass spectrometry is becoming a trend in diagnosing disease. For example, matrix assisted laser desorption ionization-time of flight mass spectrometry (MALDI-TOF MS) mass spectrometers have been used as a fast, accurate, and cost-effective way of diagnosing diseases, including microorganism identifications. In microorganism identification or disease diagnosis using mass spectrometer data, each microorganism, for instance, bacteria is represented by a mass spectrum produced by mass spectrometer, for example a MALDI-TOF device. The mass spectrum of a sample under test is compared with the mass spectrums of the reference mass spectrums stored in the database to determine the specific micro-organism. This library diagnostics method is one of most beneficial aspect comparing with other diagnostic tools, it is more economical, more time-saving, more convenient to handle, and more accurate.

Embodiments relate to optimal clustering to facilitate higher accuracy. Embodiments are based on a deconvolution concept to obtain more highly clustered sets or categories of samples. Deconvolution may be defined as a process to separate a dataset into two or more independent datasets of clusters. In embodiments, successive deconvolution is a relatively efficient way of clustering and/or way to facilitate higher diagnostic accuracy. For example, in embodiments, sets of m/z's with newly defined subcategories are used as a base of disease diagnostics for pattern matching analysis.

Cancer diagnosis using mass spectrum has been challenging because diseases are affected by many factors such as age, health condition, etc. It may be difficult to identify markers that can accurately identify the progress of a particular diseases (e.g. cancer). Mass spectrums are divided into many categories such as cancer organ types, cancer stages, patient's conditions like cholesterol levels and blood sugar levels, and patient's disease history, etc. Successful diagnosis depends on clustering efficiency with classifications or categories. Embodiments relate to finding optimal categories for cancer diagnosis.

Example FIG. 1 is a schematic view of a MALDI-TOF MS system, in accordance with embodiments. FIG. 1 shows a MALDI-TOF machine. MALDI-TOF is an analytical tool employing a soft ionization technique. Samples are embedded in a matrix and a laser pulse is fired at the mixture. The matrix absorbs the laser energy and the molecules of the mixture are ionized. The ionized molecules are then accelerated through a vacuum tube by an electrical field. Time-of flight is measured to produce the mass-to-charge ratio (m/z).

MALDI-TOF MS offers rapid identification of biomolecules such as peptides, proteins and large organic molecules with very high accuracy and sensitivity. MALDI-TOF is becoming a standard for identification of micro-organisms in clinical biology.

Example FIG. 2 shows the integrated disease diagnosis system, in accordance with embodiments. Samples may undergo a combination of process by selected modules. In the sample preparation system 301, a sample goes through a predefined and preprogrammed sequence depending on diagnosis or screening purposes in an automatic sample preparation unit 311. In embodiments, for glycan extraction, multiple processing modules may be selected, which as sample reception, protein denaturation, deglycosylation, protein removal, drying, centrifugation, solid phase extraction, and/or spotting. After sample preparation, the sample loader 312 loads the samples onto the plates 306 and are dried in a sample dryer 307.

The samples may then be provided to the MALDI-TOF MS unit 302 having an ion flight chamber 321 and/or a high voltage vacuum generator 322, in accordance with embodiments. A processing unit 323 in the MALDI-TOF MS may identify the mass/charge and its corresponding intensity. For the disease diagnostic purpose, those acquired mass and intensity data may be reorganized to set up a standard mass list, in which a concept of the center of mass where intensities are balanced and equilibrated is introduced. A standard mass to charge list is defined based upon the machine accuracy and the center of mass concept. The stored spectrum data for each laser irradiation may also be used to set up the standard mass list.

In embodiments, diagnostic unit 303 may then compare, the spectra from a patient's sample with the pre-stored spectra and analyzes the pattern difference of the two spectra. The diagnostic unit 303 may then identify the presence and progress of the disease. In embodiments, as shown in example FIG. 3, diagnostic unit 303 may be internally integrated to the MALDI-TOF MS unit 302. In embodiments, diagnostic unit 303 may be either internal or external to a mass spectrometer system. In embodiments, a diagnostic unit may be cloud based. In embodiments, a diagnostic unit may be networked to a mass spectrometer system by a local network (e.g. an intranet network), a public network (e.g. the internet), or any other network as appreciated by those skilled in the art. In embodiments, a diagnostic unit may be coupled to an artificial intelligence engine and/or to one or more processors that implement deep learning algorithms.

Example FIG. 3 illustrates an integrated disease diagnosis system where the sample preparation unit 401 and the MALDI-TOF 402 are integrated, with the diagnosis unit 403 stands apart as a separate unit, in accordance with embodiments.

Example FIG. 4 is a MALDI-TOF MS hardware diagram, in accordance with embodiments. Different types of detectors 613 are available, as appreciated by those of ordinary skill in the art. A MALDI-TOF MS system may exploit the fact that all ions 615a-c accelerated in the same electric field 605 may have the same or substantially the same kinetic energy. After leaving the electric field 605 (e.g. generated by electrodes) ions 615a-c may enter a field-free section and/or flight tube 603. Flight tube 603 may have a predetermined length 611. Ions 615a-c have different speeds depending on their mass. Large ions 615a may take more time to traverse the flight tube than smaller ions 615c.

The matrix 607 containing a sample may be irradiated by a laser 601. Both the sample molecules on the matrix 607 may be vaporized. As the matrix 607 absorbs the laser 601 and the sample becomes ionized, some of that energy is passed to the sample molecules and a number of the sample molecules become ionized 615a-c. Voltage may be applied to electrodes in a chamber containing the matrix 607, drawing the ionized molecules 615a-c to the mass spectrometer tube 603 and ultimately to detector 613.

An electrostatic field along the tube 603 of the spectrometer causes the ionized molecules 615a-c to fly down the length of the tube 603. The “time of flight” (TOF) is the time it takes the ions 615a-c to reach the detector 613 at the end of the tube 603 and depends on its mass/charge ratio (m/z) of the ionized particles 615a-c. The recorded time is converted by the spectrometer and is reported as an m/z ratio, where m is the mass of the ion in Daltons, and z is the ions' charge.

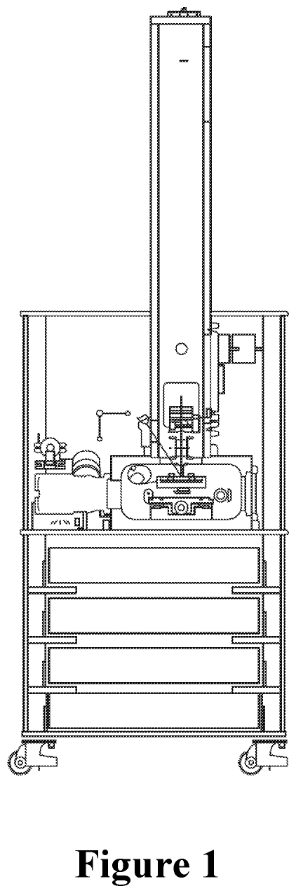

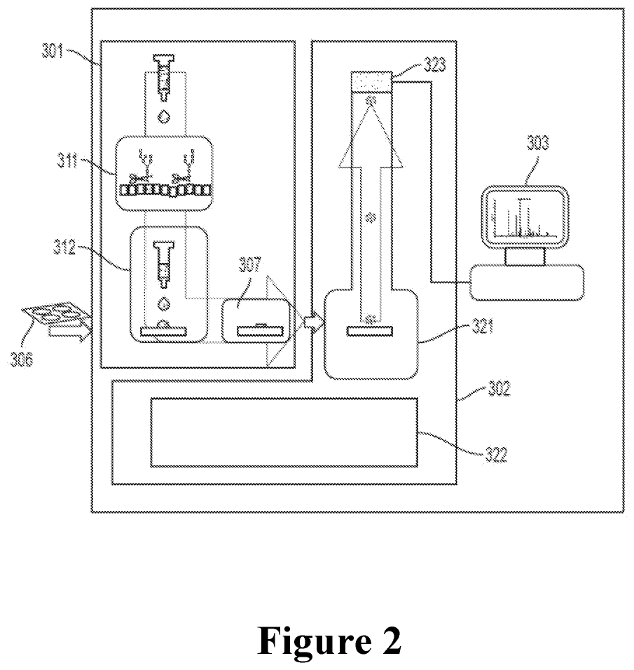

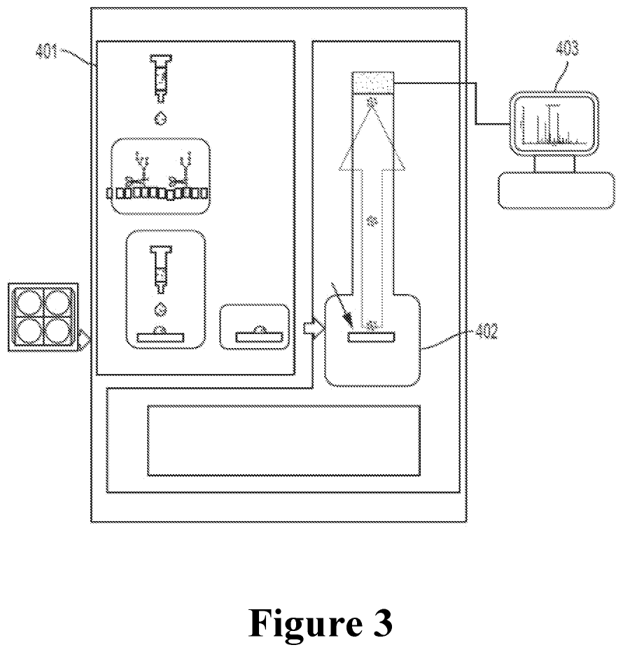

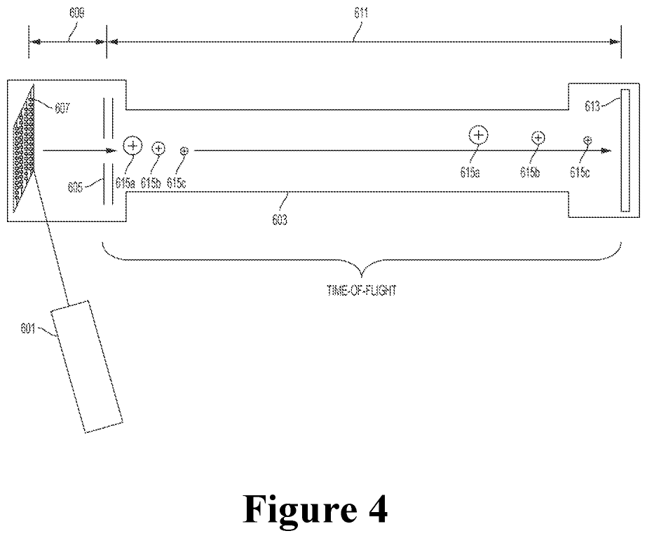

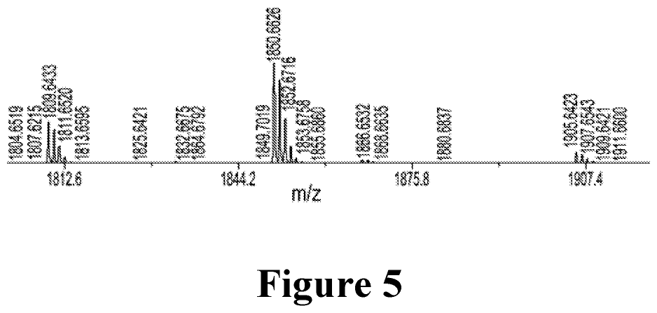

Example FIG. 5 illustrates an example MALDI-TOF mass spectra of a sample from a patient. The sample may be a bodily fluid such as saliva, urine or blood containing proteins or glycans.

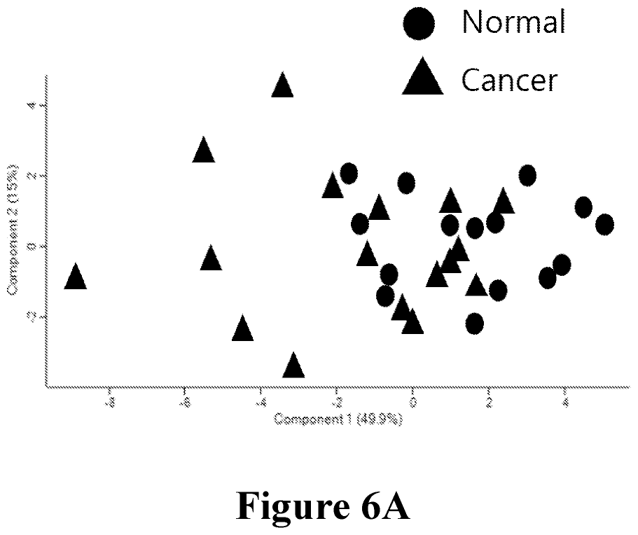

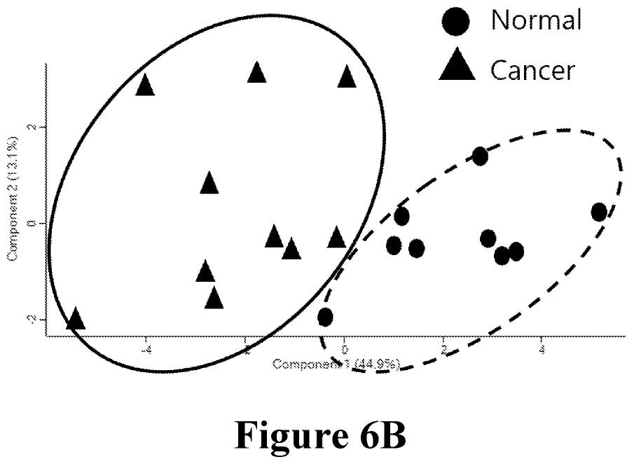

Example FIG. 6A illustrates a PCA of mass spectra of normal subjects and cancer patients, in accordance with embodiments. A PCA (Principal Component Analysis) reduces large data sets of multiple dimensionalities into smaller number of important dimensionality. The PCA shows that the samples of cancer patients and normal subjects are intermixed before optimal clustering. Example FIG. 6B the PCA after optimal clustering. It shows that the samples of cancer patients and normal subjects are clearly separated.

Example FIG. 7 illustrates a system for matching characteristic information, in accordance with embodiments. A system may include at least one processor 715. A system may include a receiving unit 701 configured to receive mass spectrometer test data of a sample using the at least one processor 715. A system may include an associating unit 703 configured to associate metadata information of a source of the sample to the mass spectrometer test data using the at least one processor 715. A system may include a selecting unit 705 configured to select a subset of a sample reference library based on the associated metadata information using the at least one processor 715. The sample reference library 711 may include a plurality of sets of mass spectrometer reference data. A matching unit 707 may be configured to match the mass spectrometer test data with at least one set of the plurality of sets of mass spectrometer reference data of the selected subset of the sample reference library 711 using the at least one processor 715. A determining unit 709 may be configured to determine characteristic information of the source based on the known characteristics of the matched mass spectrometer reference data using the at least one processor 715.

In embodiments, mass spectrometer test data may have unknown characteristics and a plurality of sets of mass spectrometer reference data has known characteristics. The sample may include biological molecules. The metadata information of the source may include information about the source of the biological molecules. The characteristic information of the source may include a biological analysis information of the source. The biological analysis information may be a medical diagnosis of at least one of a human being, an animal, a plant, or a living organism.

Example FIG. 8 illustrates a system for matching characteristic information using artificial intelligence, in accordance with embodiments. For example, artificial intelligence unit 801 may be coupled to receiving unit 701, associating unit 703, selecting unit 705, matching unit 707, determining unit 709, sample reference library 711, processor(s) 715, and/or any other unit of a system in order to optimize efficiency and/or effectiveness of a system.

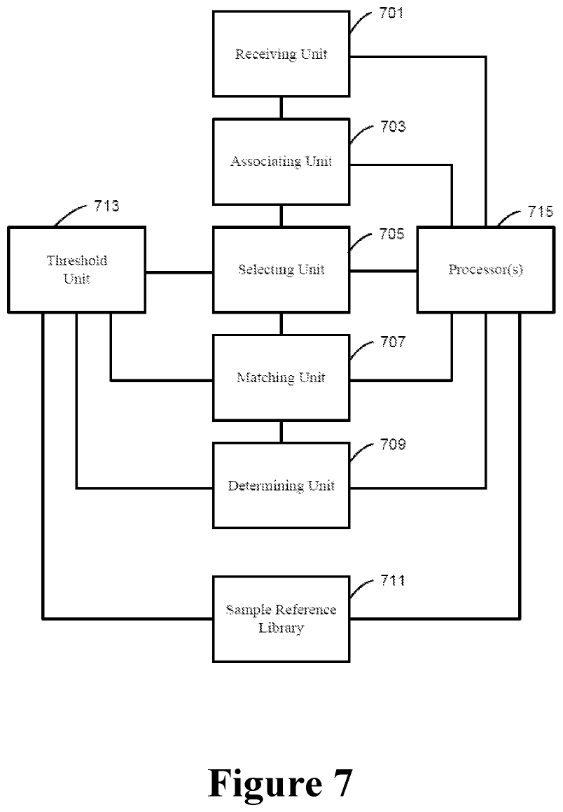

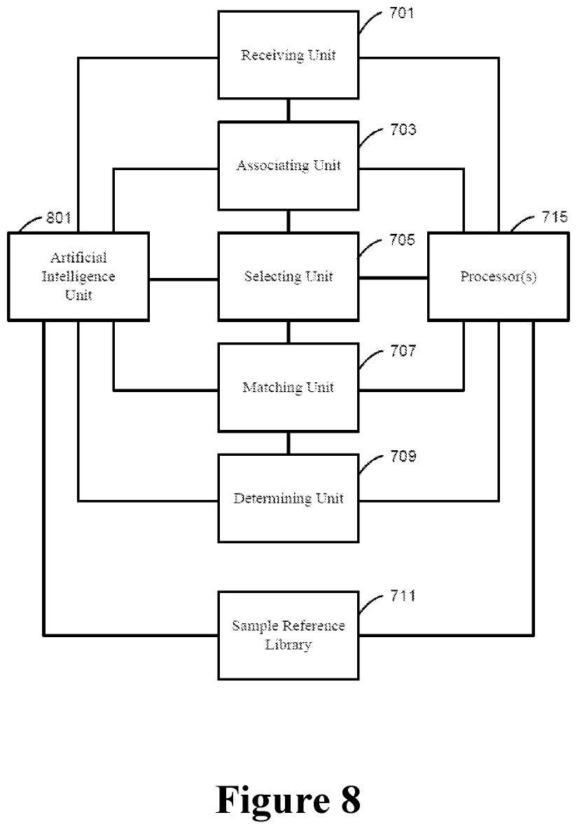

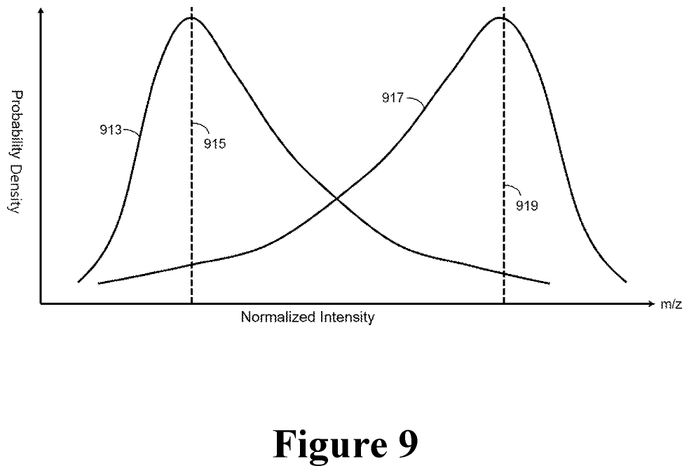

FIG. 9 illustrates an example probability density function (PDF) of the distribution of peaks in a reference database for two different subsets of metadata, in accordance with embodiments. For example, PDF 913 may be the distribution of spectrometry peaks in a reference database for cancer-free patients and PDF 917 may be the distribution of spectrometry peaks in a reference database for patients with cancer. The center of the distribution for PDF 913 is point 915, which may be expressed in units of mass-to-charge (m/z). Likewise the center of the distribution for PDF 917 is point 919. According to this simplified example, if a patient under diagnosis has a sample of their biological material analyzed by a mass spectrometer (e.g. MALDI-TOF MS), the result of that test may produce a mass spectrometry profile with a set of peaks (e.g. expressed in units of m/z). If one of those peaks is equal to or approximately at point 915, then it may be concluded that the patient under test is cancer-free. Likewise, if one of the peaks is equal to or approximately at point 915, then it may be concluded that the patient under test has cancer.

However, for example, for PDF 917 associated with cancer, this distribution of peaks in the reference database may contain more information than just the general diagnosis of cancer. In accordance with embodiments, PDF 917 may be deconvolved into multiple PDFs each associated with a different kind of cancer.

In embodiments, PDFs of a cancer patients and normal subjects at a particular m/z. Accurate classification is difficult because the two or more PDFs overlap with each other. This is due to convolution of the spectrums belonging to many categories.

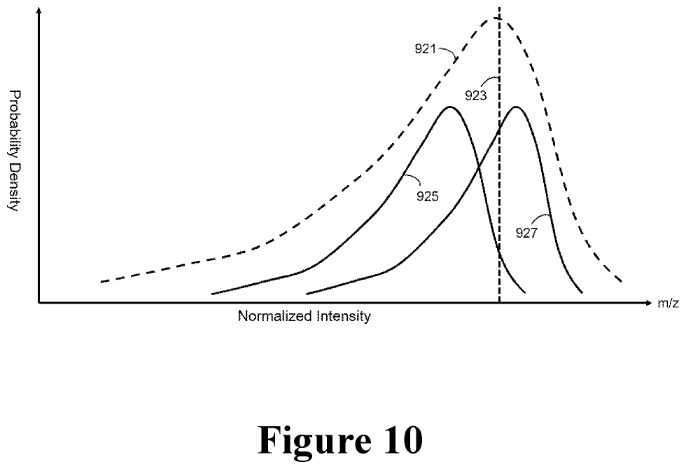

FIG. 10 illustrates embodiments, where PDF 921 may be deconvolved into multiple PDFs 925 and 927 that are attributed to sub-categories of PDF 921. For example, PDF 921 may be the distribution of peaks in a reference library for all cancers or a general category of cancers. PDFs 925 and 927 may be the distribution of peaks in a reference library for a particular type of cancer or sub-classification of this general category of cancers. For example, if PDF 921 is for lung cancer, PDF 925 may be for patients without a smoking history and PDF 927 may be for patients with a smoking history. Although the center of the distribution of PDF 921 may be point 923, the centers of distribution for PDFs 925 and 927 are different.

Cancer is only use as an example disease for the purpose of illustration and any kind of categorization, even outside of the medical field, may be applicable.

For example, without PDFs 925 and 927, mass spectrometry test data from a patient may only be compared with point 923 of PDF 921. If one of the peaks of this mass spectrometry test data is within a reasonable range of point 923, it may be generally concluded that the patient under test has cancer, but not what type of cancer. By deconvolving PDF 921 into PDFs 925 and 927, the matching system is able to use much more information from the reference library. PDFs 925 and 927 are a cluster associated with the pre-deconvoluted PDF 921. Note that the approximate or actual summation of PDFs 925 and 927 equal PDF 921, but the centers of mass of PDFs 925 and 927 are different. In this simplified example, if the mass spectrometry profile of a patient under test had a peak at approximately the center of mass of PDF 925, then it may be concluded that the patient has lung cancer, which is more information than just a comparison with PDF 921 which would only indicate the general existence of cancer.

In embodiments, the associative relationship within a cluster provide quality information. For every probability density function for a category or sub-category, if deconvolution can be performed to further define the cluster, then there is a higher accuracy in diagnosis and an improvement in resolution.

In embodiments, a deconvolution process of a cancer patient PDF may be realized. Each spectrum may be split into two or more spectrums so that at least one of the spectrums gets more distance from the spectrum of the other category (higher clustering). For example, a cancer patient category may be divided into subcategories such as different cancer stages or different types of cancers. The PDFs of subcategories are now multiple PDFs spaced apart, with different centers of mass.

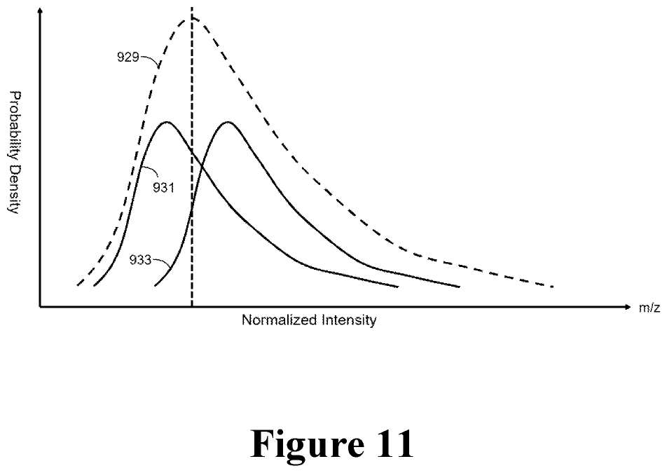

FIG. 11 illustrates embodiments, where PDF 929 represent a distribution of peaks in a reference library associated with a cancer-free diagnosis. PDF 929 may be deconvolved into PDFs 931 and 933 for two separate categories of cancer-free patient. For example, PDF 931 may be for cancer-free patients that have diabetes and PDF 933 may be for cancer-free patients that are also diabetes-free. In embodiments, any category or sub-category associated with a PDF may be deconvolved into a further sub-category of PDFs which together form a cluster. Although only two PDFs 931 and 933 are shown as being deconvolved from PDF 929, this is merely for simplification and explanatory purposes. Any number of deconvolved PDF can be derived from a pre-deconvolved PDF, in accordance with embodiments. In embodiments, a deconvolved PDF may be further deconvolved in succession to maximize and/or optimize the number of PDFs in a cluster. In embodiments, for example, a PDF of a cancer-free patient may be divided to subcategories such as age, blood sugar, or cholesterol levels. The resulting PDFs of subcategories are multiple PDFs spaced apart.

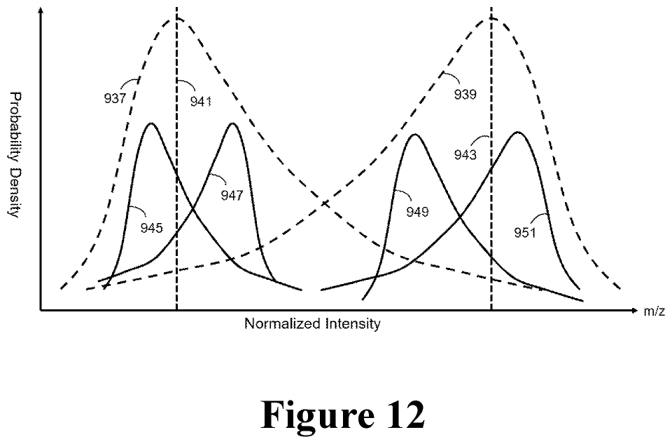

FIG. 12 illustrates an example of successive deconvolution of PDFs of peaks in a reference library for lung cancer diagnosis, in accordance with embodiments. PDFs 937 and 939 may be deconvolved PDFs from a general PDF (not shown). For example, PDF 937 may be for normal subjects and PDF 939 may be for lung cancer patients from a general PDF of cancer overall. PDF 937 may be further successively deconvolved into PDFs 945 and 947, where PDF 945 is for patients under the age 50 having lung cancer and PDF 947 is for patients over the age 50 having lung cancer. Likewise, PDF 939 may be further deconvolved into PDFs 949 and 951, wherein PDF 949 is for patients without a smoking history and PDF 951 is for patients with a smoking history. These are just examples of categories that can be deconvolved and should not be considered limiting. PDFs 945, 947, 949, 951 may be further successively deconvolved for any number of iterations which categorization or sub-categorization enhances and/or optimizes a reference library.

For example, PDFs of normal subjects of a subcategory and cancer patients after subcategorization is one of many possible ways to subdivide the deconvolutions. In embodiments, the PDFs of normal and cancer after deconvolution is spaced further apart than before deconvolution, resulting in better clustering. The area overlapped by two PDF represent the quality of clustering. The deconvolution process may be repeated until the optimal clustering is obtained. The above process of finding optimal clustering for each m/z repeated all m/z of interest. The optimal clustering is eventually used to derive a signature database that will be used to compare against an unknown patient's sample.

In embodiments, tables may be utilized for all relevant m/z's and their successive clustering results. From the set of all m/z's with optimal clustering information, a set of m/z's with optimal clustering is selected as signature database for pattern matching to accurately diagnose a cancer. One metric may be the distance between the cancer and normal clusters. The farther apart the better clustering. The areas overlapped by the normal and cancer may be used as weights for pattern matching, in accordance with embodiments.

Embodiments relate to a method of diagnosing cancer using mass spectrometry is provided. In embodiments, a method may include deconvolving the profile or the PDF of mass spectra within a category at a m/z point into two or more profiles of the category. In embodiments, a method may include repeating the deconvolution process until optimally clustered subcategories of each category at the m/z are obtained. In embodiments, a method may include repeating the optimal clustering process for other m/z's of interest. In embodiments, a method may include selecting an optimum set of m/z's to yield the best clustering. In embodiments, a method may include defining a pair of associated subcategories which shows the optimum clustering value. In embodiments, a method may include applying the optimum clustering and defining subcategorization process for the remaining data profiles until acceptable clustering outcome is achieved. In embodiments, clustered subcategories could be the existing classifications of diseases or microorganisms or the definition of a new classification.

Embodiments relate to cancer diagnostics using the mass spectrometer data. Embodiments may include deconvolving the profile or the PDF profile of mass spectra within a category at a m/z point into profiles of two or more subcategories where the category can be normal healthy people or cancer patients. Embodiments may include repeating the deconvolution process until desirable clustering subcategories of a category at the m/z are obtained, where the profile with one mode of a category being split into two profiles with each different mode and one of which being used for a higher clustering value against the other category. Embodiments may include repeating the clustering process for other m/z point of interest. Embodiments may include selecting an optimum set of m/z's to have the best and/or optimal clustering. Embodiments may include defining a pair of associated subcategories which shows the best (optimum) clustering value. Embodiments may include applying the optimum clustering and defining subcategorization process for the rest of the data until another acceptable clustering outcome is achieved or data is insufficient to perform the clustering process. In embodiments, the clustered subcategories could be the existing classifications of diseases or microorganisms or the definition of a new classification.

Embodiments relate to an apparatus and/or method that includes deconvolving a pre-deconvoluted distribution of spectrometry reference profile peaks into at least two post-deconvoluted distributions of spectrometry reference profile peaks. In embodiments, the pre-convoluted distribution of spectrometry reference profile peaks originated from spectrometry reference profiles associated with a first category. In embodiments, the at least two post-deconvoluted distributions of spectrometry reference profile peaks each originated from spectrometry reference profiles each associated with at least two different sub-categories of the first category.

Embodiments include receiving from a mass spectrometer a test mass spectrometry profile from a test on a sample. Embodiments include comparing peaks of the test mass spectrometry profile with the at least two post-deconvoluted distributions of spectrometry reference profile peaks. Embodiments include associating the test mass spectrometry profile to one of the at least two different sub-categories if at least one of the peaks of the test mass spectrometry profile is approximately the same as one of the two post-deconvoluted distributions of spectrometry reference profile peaks.

In embodiments, the mass spectrometer is comprised in a matrix assisted laser desorption ionization-time of flight mass spectrometry (MALDI-TOF MS). In embodiments, the associating the test mass spectrometry profile to one of the at least two different sub-categories enhances a medical diagnosis through clustering.

In embodiments, the first category is at least one of disease and/or microorganism. In embodiments, at least one of the at least two different sub-categories of the first category is at least one of a characteristic and/or trait of the at least one disease and/or microorganism.

In embodiments, the first category is a characteristic of a reference sample that can be categorized. In embodiments, at least one of the at least two different sub-categories of the first category is at least one of a sub-characteristic and/or sub-trait of the characteristic of the reference sample. In embodiments, the at least one of the at least two different sub-categories is associated with a source of the spectrometry reference profile. In embodiments, one of the at least two different subcategories is age of the source, gender of the source, or characteristic of the source.

In embodiments, the first category and the at least two different sub-categories of the first category are comprises in a first cluster.

In embodiments, the pre-deconvoluted distribution of spectrometry reference profile peaks originated from spectrometry reference profiles associated with a second category. In embodiments, the at least two post-deconvolution distributions of spectrometry reference profile peaks each originated from spectrometry reference profiles each associated with at least two difference sub-categories of the second category. In embodiments, the second category and the at least two different sub-categories of the second category comprises a second cluster.

In embodiments, peaks of the pre-convoluted distribution of spectrometry reference profile peaks and the at least two post-deconvolution distributions of spectrum reference profile peaks are in units of mass-to-charge.

Embodiments include deconvolving at least one of the two post-deconvoluted distributions of spectrometry reference profile peaks into at least two secondary-post-deconvoluted distributions of spectrometry reference profile peaks. In embodiments, the at least two secondary-post-deconvoluted distributions of spectrometry reference profile peaks are each associated with at least two different secondary-sub-categories of at least one of the two different sub-categories of the first category. In embodiments, the first category, the at least two different sub-categories, and the at least two different secondary-sub-categories comprises a first cluster.

Embodiments include performing at least one subsequent deconvolving operations on the first cluster. In embodiments, the performing at least one subsequent deconvolving operations on the first cluster comprises an optimal number of deconvolving operations to optimize the first cluster.

In embodiments, the apparatus and/or method is performed on at least of a server and/or by cloud computing. In embodiments, the apparatus and/or method is performed using at least one of artificial intelligence and/or at least one deep learning algorithm.

Although the above-described embodiments are described based on a series of steps or flowcharts, this does not limit the time series order of the invention and may be performed simultaneously or in a different order as necessary. In addition, in the above-described embodiment, each component (for example, a unit, a module, etc.) constituting the block diagram may be implemented as a hardware device or software, and a plurality of components are combined into one hardware device or software. The above-described embodiments may be implemented in the form of program instructions that may be executed by various computer components, and may be recorded in a computer-readable recording medium. The computer-readable recording medium may include program instructions, data files, data structures, etc. alone or in combination. Examples of computer-readable recording media include magnetic media such as hard disks, floppy disks, and magnetic tape, optical recording media such as CD-ROMs, DVDs, and magneto-optical media such as floptical disks, media) and hardware devices specifically configured to store and execute program instructions, such as ROM, RAM, flash memory, and the like. The hardware device may be configured to operate as one or more software modules to perform the process according to the invention, and vice versa.

It will be obvious and apparent to those skilled in the art that various modifications and variations can be made in the embodiments disclosed. This, it is intended that the disclosed embodiments cover the obvious and apparent modifications and variations, provided that they are within the scope of the appended claims and their equivalents.

What is claimed is:

1. A method comprising:
deconvolving a pre-deconvoluted distribution of spectrometry reference profile peaks into at least two post-deconvoluted distributions of spectrometry reference profile peaks,
wherein the pre-convoluted distribution of spectrometry reference profile peaks originated from spectrometry reference profiles associated with a first category, and
wherein the at least two post-deconvoluted distributions of spectrometry reference profile peaks each originated from spectrometry reference profiles each associated with at least two different sub-categories of the first category.

2. The method of claim 1, comprising:
receiving from a mass spectrometer a test mass spectrometry profile from a test on a sample;
comparing peaks of the test mass spectrometry profile with the at least two post-deconvoluted distributions of spectrometry reference profile peaks; and
associating the test mass spectrometry profile to one of the at least two different sub-categories if at least one of the peaks of the test mass spectrometry profile is approximately the same as one of the two post-deconvoluted distributions of spectrometry reference profile peaks.

3. The method of claim 2, wherein the mass spectrometer is comprised in a matrix assisted laser desorption ionization-time of flight mass spectrometry (MALDI-TOF MS).

4. The method of claim 2, wherein the associating the test mass spectrometry profile to one of the at least two different sub-categories enhances a medical diagnosis through clustering.

5. The method of claim 1, wherein the first category is at least one of disease and/or microorganism.

6. The method of claim 5, wherein at least one of the at least two different sub-categories of the first category is at least one of a characteristic and/or trait of the at least one disease and/or microorganism.

7. The method of claim 1, wherein the first category is a characteristic of a reference sample that can be categorized.

8. The method of claim 7, wherein at least one of the at least two different sub-categories of the first category is at least one of a sub-characteristic and/or sub-trait of the characteristic of the reference sample.

9. The method of claim 8, wherein:
the at least one of the at least two different sub-categories is associated with a source of the spectrometry reference profile; and
one of the at least two different subcategories is age of the source, gender of the source, or characteristic of the source.

10. The method of claim 1, wherein the first category and the at least two different sub-categories of the first category are comprises in a first cluster.

11. The method of claim 1, wherein:
the pre-deconvoluted distribution of spectrometry reference profile peaks originated from spectrometry reference profiles associated with a second category; and
the at least two post-deconvolution distributions of spectrometry reference profile peaks each originated from spectrometry reference profiles each associated with at least two difference sub-categories of the second category.

12. The method of claim 11, wherein the second category and the at least two different sub-categories of the second category comprises a second cluster.

13. The method of claim 1, wherein peaks of the pre-convoluted distribution of spectrometry reference profile peaks and the at least two post-deconvolution distributions of spectrum reference profile peaks are in units of mass-to-charge.

14. The method of claim 1, comprising:
deconvolving at least one of the two post-deconvoluted distributions of spectrometry reference profile peaks into at least two secondary-post-deconvoluted distributions of spectrometry reference profile peaks,
wherein the at least two secondary-post-deconvoluted distributions of spectrometry reference profile peaks are each associated with at least two different secondary-sub-categories of at least one of the two different sub-categories of the first category.

15. The method of claim 14, wherein the first category, the at least two different sub-categories, and the at least two different secondary-sub-categories comprises a first cluster.

16. The method of claim 15, comprising performing at least one subsequent deconvolving operations on the first cluster.

17. The method of claim 16, wherein the performing at least one subsequent deconvolving operations on the first cluster comprises an optimal number of deconvolving operations to optimize the first cluster.

18. The method of claim 1, wherein the method is performed on at least of a server and/or by cloud computing.

19. The method of claim 1, wherein the method is performed using at least one of artificial intelligence and/or at least one deep learning algorithm.

20. An apparatus configured to:
deconvolve a pre-deconvoluted distribution of spectrometry reference profile peaks into at least two post-deconvoluted distributions of spectrometry reference profile peaks,
wherein the pre-convoluted distribution of spectrometry reference profile peaks originated from spectrometry reference profiles associated with a first category, and
wherein the at least two post-deconvoluted distributions of spectrometry reference profile peaks each originated from spectrometry reference profiles each associated with at least two different sub-categories of the first category.
